(19) United States
(12) Patent Application Publication
(10) Pub. No.: US 2014/0155429 A1
(43) Pub. Date: Jun. 5, 2014

(54) Treatment of Cognitive Disorders with Certain Alpha-7 Nicotinic Acid Receptor Agonists in Combination with Nicotine

(71) Applicant: EnVivo Pharmaceuticals, Inc., Watertown, MA (US)

(72) Inventor: Gerhard Koenig, Newton, MA (US)

(73) Assignee: EnVivo Pharmaceuticals, Inc., Watertown, MA (US)

(21) Appl. No.: 14/116,016

(22) PCT Filed: May 8, 2012
(86) PCT No.: PCT/US2012/036873

§ 371 Date: Jan. 28, 2014

Related U.S. Application Data
(60) Provisional application No. 61/483,873, filed on May 9, 2011.

(51) Int. Cl. C07D 453/02; A61K 31/439; A61K 31/4439

(52) U.S. Cl. CPC C07D 453/02; A61K 31/4439; A61K 31/439
USPC 514/305

(57) ABSTRACT

A method for improving cognition comprising co-administering to a subject an alpha 7 agonist, or a pharmaceutically acceptable salt thereof and a tobacco-free nicotine dosage is described together with related compositions.

### CROSS REFERENCE TO RELATED APPLICATION

This application claims the benefit of priority from U.S. Provisional Application No. 61/483,873, filed May 9, 2011. The foregoing related application, in its entirety, is incorporated herein by reference.

### BACKGROUND OF THE INVENTION

Nicotinic acetylcholine receptors (nAChR) form a family of ion channels activated by acetylcholine. Functional receptors contain five subunits, and there are numerous receptor subtypes. Studies have shown that central nicotinic acetylcholine receptors are involved in learning and memory. Nicotinic acetylcholine receptors of the alpha7 subtype are prevalent in the hippocampus and cerebral cortex.

WO 03/055878, published in 2003, describes a variety of agonists of the alpha7 nAChR said to be useful for improving cognition. WO 03/055878 suggests that certain agonists of the alpha7 nAChR are useful for improving perception, concentration, learning or memory, especially after cognitive impairments like those occurring for example in conditions/diseases/syndromes such as mild cognitive impairment, age-associated learning and memory impairments, age-associated memory loss, Alzheimer's disease (AD), schizophrenia and certain other cognitive disorders.

In the years following this publication there have been numerous additional publications that have continued to develop the area related to therapy for improving cognition; however, there is no standard of treatment on the market today that would satisfy the current need for effective treatment of cognitive disorders. As such, there is a desperate need for additional therapies useful for improving cognition and/or treating cognitive disorders.

### SUMMARY OF THE INVENTION

The present invention provides methods for improving cognition and/or treating cognitive disorders comprising co-administering to a subject an alpha7 agonist, or a pharmaceutically acceptable salt thereof, and a tobacco-free nicotine dosage (TFN). In particular, the combination of an alpha7 agonist of the invention, e.g., a compound of formula (I), with a TFN has been found to more effectively improve cognition and/or treat cognitive disorders than administration of either of them alone. Furthermore, the present invention provides specific compositions, e.g., pharmaceutical compositions, comprising an alpha7 agonist and a TFN.

Accordingly, one aspect the invention provides a method for improving cognition and/or treating a cognitive disorder comprising co-administering to a subject a therapeutically effective amount of (1) a tobacco-free nicotine dosage; and (2) a compound of formula (I), or a pharmaceutically acceptable salt thereof,

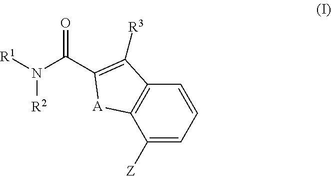

wherein

R¹ is 1-azabicyclo[2.2.2]oct-3-yl,

R² is hydrogen or (C₁-C₆)alkyl,

R³ is hydrogen, halogen or (C₁-C₆)alkyl,

A is oxygen or sulfur, and

Z is halogen, formyl, carbamoyl, cyano, trifluoromethyl, trifluoromethoxy, nitro, amino, formamido, acetamido, (C₁-C₆)alkyl, (C₁-C₆)alkyoxy, (C₁-C₆)alkylthio, (C₁-C₆)alkylamino, heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino)methyl. In one embodiment, the compound of formula (I) is (R)-7-chloro-N-(quinuclidin-3-yl)benzo[b]thiophene-2-carboxamide.

In another aspect, the invention provides a pharmaceutical composition comprising (R)-7-chloro-N-(quinuclidin-3-yl)benzo[b]thiophene-2-carboxamide or a pharmaceutically acceptable salt thereof, and a tobacco-free nicotine dosage (TFN).

In yet another aspect, the invention provides a daily unit dosage pharmaceutical composition comprising 0.03 to 1.0 mg of (R)-7-chloro-N-(quinuclidin-3-yl)benzo[b]thiophene-2-carboxamide or a pharmaceutically acceptable salt thereof, a TFN, and a pharmaceutically acceptable carrier.

In yet another aspect, the invention provides a packaged pharmaceutical comprising a package containing a unit dosage pharmaceutical composition comprising (R)-7-chloro-N-(quinuclidin-3-yl)benzo[b]thiophene-2-carboxamide or a pharmaceutically acceptable salt thereof, and a transdermal patch comprising a tobacco-free nicotine dosage.

### DETAILED DESCRIPTION OF THE INVENTION

The present invention provides an unexpectedly superior combination of an alpha7 agonist, e.g., a compound of formula (I), and a tobacco-free nicotine dosage (TFN), as well as methods for co-administering this combination, and preparing the combination to treat cognitive disorders and/or improving cognition, including one or more symptoms of Alzheimer's disease.

Furthermore, the present invention, including compounds, methods, and pharmaceutical compositions will be described with reference to the following definitions that, for convenience, are set forth below. Unless otherwise specified, the below terms used herein are defined as follows:

### I. DEFINITIONS

Reference herein to a “combination” is to the co-administration of a component (1) and component (2), each as defined herein, either as a composition or separately, e.g., by different routes of administration. In addition, another component may also be combined with components (1) and (2) to afford additional unique and beneficial combination therapies

“Co-administration” as used herein describes the purposeful election to administer at least two individual components (e.g., an alpha7 agonist and a TFN) based on a prior understanding of the utility of the administration, and a desire to benefit from the added or synergistic effect of the administration of the components in combination. Such administration is in contrast to the incidental administration of, for example, sub-optimal doses delivered through cigarette consumption or nicotine patch application in response to craving reflex, wherein such doses are considered sub-optimal in that they contain impurities, e.g., toxins, or are at unregulated doses that are outside the therapeutically effective treatment ranges considered herein.

Moreover, the individual components can be administered together as a composition, if the route of administration is the same. Thus the invention further provides a composition comprising: (1) a compound of the invention, e.g., a compound formula (1) as defined, or a pharmaceutically acceptable salt thereof, and (2) a tobacco-free nicotine dosage, wherein the amount of the first component alone and the amount of the second component alone are each insufficient to achieve the combined therapeutically effective level of improving cognition; and a pharmaceutically acceptable vehicle, diluent, or carrier.

“Co-administration” also includes administering each of components (1) and (2) separately but as part of the same therapeutic treatment program or regimen, and it is contemplated that separate administration of each component, at different times and by different routes, will sometimes be recommended. Thus, the two components need not necessarily be administered at essentially the same time or in any particular order. In an embodiment, administration is timed so that the peak pharmacokinetic effect of one component coincides with the peak pharmacokinetic effect for the other.

The expression “therapeutically effective,” as used herein, describes amounts or doses of a compound useful for the treatment of a disorder, alone or in combination with other compounds/compositions, that are therapeutically effective for the purpose for which they were intended and achieve a therapeutic effect, e.g., improving cognition. In certain embodiments, to achieve a therapeutic effect, doses or amounts are provided by a well-regulated, or well-designed regimen of administration. As such, therapeutically effective amounts or doses include amounts or doses that would not otherwise be therapeutically effective alone (i.e., in the absence of the combinations of the present invention), or what might otherwise be referred to as a subclinical dose.

As used herein, the expression “a tobacco-free nicotine dosage” or “TFN,” which are used interchangeably herein, describe a composition comprising nicotine, substantially free of the impurities that are associated with tobacco or any one of the 599 officially FDA recognized and approved additives that are solely associated with processed tobacco, e.g., such as tobacco in a cigarette (in comparison to, for example, ingredients/additives that are also in other nicotine sources), which include, but are not limited to, any one of the components of a cigarette (e.g., tobacco, menthol, or cellulose fiber), or any one of the over 4000 toxins created by burning tobacco, e.g., a cigarette. As used herein, the expression, “substantially free of is used with reference to the presence of these impurities, components, additives, or toxins associated with an average portion of tobacco consumed in one sitting, e.g., an average cigarette, wherein the amounts present in a TFN of the invention are no more than 5%, e.g., 4%, e.g., 3%, e.g., 2%, e.g., 1%, e.g., 0.5%, e.g., 0.1% by weight as compared with the amount by weight present in an average tobacco portion, e.g., an average cigarette. Some examples of a tobacco-free nicotine dosage include, without limitation, the compositions included in nicotine gum, nicotine patches, nicotine inhalers, and nicotine nasal sprays. A tobacco-free nicotine dosage may include 7.5% to 99% by weight nicotine, 10% to 75% by weight nicotine, or 15% to 60% by weight nicotine. A TFN of the present invention is intended to deliver a blood concentration of nicotine greater than zero, but less than 1000 nmol/L, e.g., less than 500 nmol/L, e.g., less than 450 nmol/L, e.g., less than 400 nmol/L, e.g., less than 350 nmol/L, e.g., less than 300 nmol/L, e.g., less than 250 nmol/L, e.g., less than 200 nmol/L, e.g., less than 150 nmol/L, e.g., less than 100 nmol/L, e.g., less than 50 nmol/L, e.g., less than 25 nmol/L. In certain embodiments, a TFN of the present invention is intended to deliver a blood concentration of nicotine within the range of 25 to 444 nmol/L, e.g., 200 to 210 nmol/L.

As used herein, “cognition” or “cognitive function” refers to the process of thought. A cognitive disorder refers to a deficiency in cognition: for example, a subnormal functioning in one or more cognitive aspects such as memory, intellect, or learning and logic ability, in a particular individual relative to other individuals within the same general age population. Also, any reduction in any particular individual's functioning in one or more cognitive aspects, for example as occur in age-related cognitive decline. Examples of disorders that comprise as a symptom a deficiency in cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; Alzheimer's related dementia; delirium; amnestic disorder; post-traumatic stress disorder; mental retardation; a learning disorder, for example reading disorder, mathematics disorder, post operative cognitive decline, or a disorder of written expression; attention-deficit/hyperactivity disorder; age-related cognitive decline; and any cognitive impairments resulting from chemical dependency.

As used herein, the terms “subject”, and “patient” are used interchangeably. The terms “subject” and “patient” refer to an animal (e.g., a bird such as a chicken, quail or turkey) or a mammal including non-primates (e.g., a cow, pig, horse, sheep, rabbit, guinea pig, rat, cat, dog, and mouse) and primates (e.g., a monkey, chimpanzee and a human). In a particular embodiment, the subject is a human.

As used herein, “alkyl” refers to a linear or branched saturated or unsaturated aliphatic C₁-C₆ hydrocarbon, unless some other number of carbon atoms is specified. Unsaturation in the form of a double or triple carbon-carbon bond may be internal or terminally located and, in the case of a double bond, both cis and trans isomers are included. An optionally substituted alkyl can be independently substituted with one or more substituents selected from halogen, e.g., F, oxo, OH, (C₁-C₄)alkoxy, (C₃-C₆)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH₂, —S(O)₂NH(C₁-C₄)alkyl, —S(O)₂N[(C₁-C₄)alkyl(C₁-C₄)alkyl]. Examples of alkyl groups include, but are not limited to, methyl, ethyl, n-propyl, isopropyl, t-butyl, n-pentyl, n-hexyl, isobutyl, neopentyl, cis- and trans-2-butenyl, isobutenyl and propargyl. C₁-C₄ alkyl is the subset of alkyl limited to a total of up to 4 carbon atoms.

In each case in which a size range for the number of atoms in a ring or chain is disclosed, all subsets are disclosed. Thus, Cₓ-C_{y} includes all subsets, e.g., C₁-C₄ includes C₁-C₂, C₂-C₄, and C₁-C₃ as well as C₁, C₂, C₃ and C₄.

As used herein “alkoxy” refers to an alkyl-O— group wherein alkyl is as defined above. C₁-C₆ alkoxy is the subset of alkyl-O— where the subset of alkyl is limited to a total of up to 6 carbon atoms. Examples of alkoxy groups include methoxy, trifluoromethoxy, ethoxy, trifluoroethoxy, and propoxy.

As used herein, “alkylthio” refers to an alkyl-S— group wherein alkyl is as defined above. C₁-C₆ alkylthio is the subset of alkyl-S— where the subset of alkyl is limited to a total of up to 6 carbon atoms.

As used herein, “alkylamino” refers to alkyl-NH— wherein alkyl is as defined above.

As used herein, the term “aryl” means a mono- or polycyclic hydrocarbon, containing from 6 to 15 carbon atoms, in which at least one ring is aromatic. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Aryl groups included in compounds of this invention may be optionally substituted with one or more substituents. In one embodiment, the aryl group is a monocyclic ring, wherein the ring comprises 6 carbon atoms. An optionally substituted aryl can be independently substituted with one or more substituents selected from halogen, CF_{3,} CN, OH, (C₁-C₄)alkoxy, (C₁-C₄)alkylthio, (C₃-C₇)cycloalkylthio, (C₃-C₇)cycloalkyloxy, aryloxy, (C₁-C₄)alkoxy(C₁-C₄)alkyloxy, hetero(C₃-C₇)cycloalkyloxy, heteroaryloxy, —OC(O)R_{a,} —OC(O)NHRₐ, —OC(O)N(Rₐ)(R_{b}), —S(O)Rₐ, —NHRₐ, —N(Rₐ)(R_{b}), —NHC(O)Rₐ, —N(Rₐ)C(O)R_{b}, —NHC(O)ORₐ, —N(Rₐ)C(O)OR_{b}, —N(Rₐ)—C(O)—NH(R_{b}), —N(Rₐ)—C(O)—N(R_{b})₂, —C(O)NH₂, —C(O)NHRₐ, —C(O)N(Rₐ)(R_{b}), —CO₂H, —CO₂Rₐ, —CORₐ and R_{c} wherein Rₐ, R_{b} and R_{b} are independently chosen from (C₁-C₆)alkyl, (C₁-C₄)alkoxy(C₁-C₄)alkyl, —CH₂CH₂OH, —CH₂CH₂OMe, (C₃-C₇)cycloalkyl, (C₃-C₇)cycloalkyl(C₁-C₄)alkyl, aryl, arylalkyl, heteroaryl, heteroarylalkyl, hetero(C₃-C₇)cycloalkyl, and hetero(C₃-C₇)cycloalkyl(C₁-C₄)alkyl, each of which is optionally and independently substituted with up to three groups selected from halogen, (C₁-C₆)alkyl, (C₃-C₇)cycloalkyl, (C₃-C₇)cycloalkyloxy, (C₃-C₇)cycloalkylalkoxy, CN, CHF₂, CF₃, CH₂CF₃, NHMe, NMe₂, piperidinyl, morpholinyl, N-Me-piperazinyl, piperazinyl, OCF₃, OCHF₂, OCH₂CF₃, SMe, each of which are attached via carbon-carbon or carbon-nitrogen or carbon-oxygen single bonds, and none of which are substituted; or Rₐ and R_{b} taken together with the atom(s) to which they are attached form a 5-6 membered ring. In other cases, an optionally substituted aryl can be independently substituted with one or more substituents selected from halogen CF_{3,} CN, (C₁-C₆)alkyl, (C₃-C₇)cycloalkyl, (C₁-C₄)alkoxy, (C₃-C₇)cycloalkylthio, (C₃-C₇)cycloalkyloxy, hetero(C₃-C₇)cycloalkyl, C(O)NH₂, —C(O)NHRₐ, —C(O)N(Rₐ)(R_{b}) wherein Rₐ and R_{b} are defined as above. In further cases, an optionally substituted aryl can be independently substituted with one or more substituents selected from halogen CF_{3,} CN, (C₁-C₆)alkyl, (C₃-C₇)cycloalkyl, (C₃-C₇)cycloalkyloxy, and hetero(C₃-C₇)cycloalkyl.

As used herein “cycloalkyl” is a C₃-C₈ cyclic non-aromatic hydrocarbon which may contain a single double bond. An optionally substituted cycloalkyl can be independently substituted with one or more substituents selected from F, oxo, OH, (C₁-C₆)alkyl, (C₁-C₄)alkoxy, —(C₃-C₇)cycloalkyl, (C₃-C₇)cycloalkylalkyl, (C₃-C₇)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH₂, —S(O)₂NH(C₁-C₄)alkyl, —S(O)₂N[(C₁-C₄)alkyl(C₁-C₄)alkyl]. In other cases, an optionally substituted cycloalkyl can be independently substituted with one or more substituents selected from F, oxo, (C₁-C₄)alkoxy, (C₃-C₇)cycloalkyl, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH(C₁-C₄)alkyl, —S(O)₂N[(C₁-C₄)alkyl(C₁-C₄)alkyl]. In further cases, an optionally substituted cycloalkyl can be independently substituted with one substituent selected from oxo, OH, (C₁-C₆)alkyl, (C₁-C₄)alkoxy, (C₃-C₇)cycloalkylalkyl, (C₃-C₇)cycloalkyloxy, (C₁-C₄)alkylthio, (C₃-C₆)cycloalkylthio-, —C(O)NH₂, —C(O)NH(C₁-C₄)alkyl, —C(O)N[(C₁-C₄)alkyl(C₁-C₄)alkyl], (C₁-C₄ alkyl)-C(O)—, (C₁-C₄)alkylsulfonyl-, —S(O)₂NH₂, —S(O)₂NH(C₁-C₄)alkyl, —S(O)₂N[(C₁-C₄)alkyl(C₁-C₄)alkyl]. Examples of cycloalkyl groups include cyclopropyl, cyclobutyl, cyclopentyl, cyclohexyl, cyclopentenyl and cyclohexanonyl.

As used herein, “halogen” refers to F, CI, Br or I. In particular embodiments, halogens are F, Cl and Br.

As used herein, the term “halo-substituted alkyl” or “haloalkyl” refers to an alkyl as defined herein which is substituted by one or more halogen atoms, or halo groups as defined herein. The haloalkyl can be monohaloalkyl, dihaloalkyl, polyhaloalkyl, or perhaloalkyl. For example, a monohaloalkyl contains one iodo, bromo, chloro or fluoro within the alkyl group. For multiple substituted haloalkyls, the halo atoms may be the same or a combination of different halo groups within the alkyl. Typically the polyhaloalkyl contains up to 6, or 4, or 3, or 2 halo groups. In a preferred embodiment, halo-substituted alkyl groups have about 1 to 6 carbons, or 1-3 carbons. Non-limiting examples of haloalkyl include fluoromethyl, difluoromethyl, trifluoromethyl, chloromethyl, dichloromethyl, trichloromethyl, pentafluoroethyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms.

As used herein, the term “heteroaryl” refers to a 5-14 membered monocyclic- or bicyclic- or tricyclic-aromatic ring system, having 1 to 8 heteroatoms selected from N, O or S. Typically, the heteroaryl is a 5-10 membered ring system (e.g., 5-7 membered monocycle or an 8-10 membered bicycle). A 5-7 membered monocyclic ring system preferably contains 1 to 3 heteroatoms each independently selected from O, N, or S. Exemplary heteroaryl groups include, but are not limited to 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, or 5-pyrimidinyl.

As used herein, “nicotine” refers to both nicotine (1-methyl-2-(3-pyridyl)pyrrolidone) and its derivatives, including any of the conventional nicotine compounds, including nicotine, nicotine resin complex, nicotine free base, pharmaceutically acceptable salts thereof, as well as mixtures of free base and salt forms. In one embodiment, the pharmaceutically acceptable salt of nicotine is the hydrogen tartrate salt, or nicotine dihydrogen ditartrate.

As used herein the term “dose” is the amount of active pharmaceutical ingredient (API) administered to a subject. For example 1 mg means 1 mg of API was orally administered to each subject each day.

In certain embodiments, where the “API” or “Active Pharmaceutical Ingredient” is defined as a compound of formula (I), e.g., (R)-7-chloro-N-(quinuclidin-3-yl)benzo[b]thiophene-2-carboxamide, such language is intended to include pharmaceutically acceptable salts, solvates, or solvates of pharmaceutically acceptable salts. For example, the recited compound (R)-7-chloro-N-(quinuclidin-3-yl)benzo[b]thiophene-2-carboxamide includes either (R)-7-chloro-N-(quinuclidin-3-yl)benzo[b]thiophene-2-carboxamide hydrochloride, (R)-7-chloro-N-(quinuclidin-3-yl)benzo[b]thiophene-2-carboxamide, (R)-7-chloro-N-(quinuclidin-3-yl)benzo[b]thiophene-2-carboxamide hydrochloride monohydrate or (R)-7-chloro-N-(quinuclidin-3-yl)benzo[b]thiophene-2-carboxamide hydrochloride solvate. Where solvate represents a stoichiometric ratio of 0.1 to 10 molecules of solvent compared to (R)-7-chloro-N-(quinuclidin-3-yl)benzo[b]thiophene-2-carboxamide hydrochloride or (R)-7-chloro-N-(quinuclidin-3-yl)benzo[b]thiophene-2-carboxamide. Solvent molecules include but are not limited to water, methanol, 1,4 dioxane, ethanol, iso-propanol or acetone. In some cases water is the preferred solvate.

### II. COMPOUNDS OF THE INVENTION

The compositions of the invention useful in the methods described herein are comprised of an alpha7 agonist in combination with a tobacco-free nicotine dosage, both of which are described more completely herein below.

### Alpha7 Agonists of the Invention

The compositions of the present invention comprise an alpha7 agonists. The expression “alpha7 agonist”, or “α7 agonist,” is an art-recognized expression that describes any agonist of the alpha7 nAChR, or alpha7 nicotinic acetylcholine receptor. In the compositions and combinations described herein, the alpha7 agonist is not nicotine.

In one embodiment, the alpha7 agonist is a compound of formula (I), or a pharmaceutically acceptable salt thereof,

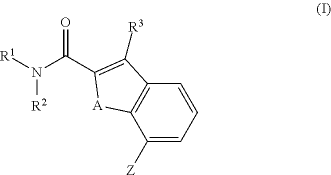

wherein

R¹ is 1-azabicyclo[2.2.2]oct-3-yl,

R² is hydrogen or (C₁-C₆)alkyl,

R³ is hydrogen, halogen or (C₁-C₆)alkyl,

A is oxygen or sulfur, and

Z is halogen, formyl, carbamoyl, cyano, trifluoromethyl, trifluoromethoxy, nitro, amino, formamido, acetamido, (C₁-C₆)alkyl, (C₁-C₆)alkyoxy, (C₁-C₆)alkylthio, (C₁-C₆)alkylamino, heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino)methyl.

In certain embodiments of the invention wherein the alpha7 agonist is a compound of formula (I), R² is hydrogen, R³ is hydrogen, A is sulfur, and Z is halogen, formyl, carbamoyl, cyano, trifluoromethyl, trifluoromethoxy, nitro, amino, formamido, acetamido, (C₁-C₆)alkyl, (C₁-C₆)alkyoxy, (C₁-C₆)alkylthio, or (C₁-C₆)alkylamino.

In certain embodiments of the invention wherein the alpha7 agonist is a compound of formula (I), Z is heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino). In certain other embodiments, Z is halogen, cyano, trifluoromethyl, trifluoromethoxy, methyl, ethyl, methoxy, or ethoxy.

In a particular embodiment of the invention, the compound of formula (I) is (R)-7-chloro-N-(quinuclidin-3-yl)benzo[b]thiophene-2-carboxamide, or a salt or solvate thereof, e.g., a hydrochloride salt, a monohydrate, or a combination thereof.

Certain other agonists of the alpha7 nAChR may also be used in combination with a tobacco-free nicotine dosage in the methods described herein. Some alpha7 agonists which may be used include those shown in WO 2004/029050, WO 2006/065233, US 2005/0245531, WO 2005/092890, WO 2007/038367, WO 2005/092890, WO 2002/7038367, and U.S. Pat. No. 6,953,855, the contents of which are herein incorporated by reference. In certain embodiments of the invention, the alpha7 agonist may be selected from well-known alpha7 agonists, for example N-[2-(pyridin-3-ylmethyl)-1-azabicyclo[2.2.2]oct-3-yl]-1-benzofuran-2-carboxamide (TC-5619); (S)—N-(quinuclidin-3-yl)-1H-indazole-3-carboxamide; (R)—N-(quinuclidin-3-yl)-1H-indazole-3-carboxamide; (S)-3-(6-(1H-indol-5-yl)pyridazin-3-yloxy)quinuclidine; and (R)-3-(6-(1H-indol-5-yl)pyridazin-3-yloxy)quinuclidine, and enantiomers and pharmaceutically acceptable salts thereof. In a particular embodiment, the alpha7 agonist may be selected from N-[2-(pyridin-3-ylmethyl)-1-azabicyclo[2.2.2]oct-3-yl]-1-benzofuran-2-carboxamide (TC-5619); (S)—N-(quinuclidin-3-yl)-1H-indazole-3-carboxamide; and (R)-3-(6-(1H-indol-5-yl)pyridazin-3-yloxy)quinuclidine, and enantiomers and pharmaceutically acceptable salts thereof, including, for example, an HCl salt form (such as In certain embodiments, the alpha7 agonist of the invention is not TC-5619. In particular embodiments, the alpha7 agonist of the invention is not TC-5619, when the TFN is a nicotine patch.

### III. TOBACCO-FREE NICOTINE DOSAGE (TFN)

The compositions of the present invention also comprise a tobacco-free nicotine dosage or TFN. As noted hereinabove, a TFN of the present invention is substantially free of the impurities otherwise associated with tobacco, including any one of the components of a cigarette (e.g., tobacco, menthol, or cellulose fiber), any one of the 599 officially FDA recognized and approved additives, or any one of the over 4000 toxins created by burning a cigarette.

Although nicotine, 1-methyl-2-(3-pyridyl)pyrrolidone, is a well-known ingredient in tobacco, it may be isolated in its pure state, or synthetically prepared through well-known chemical synthesis (e.g., see Tetrahedron, Volume 63, Issue 34, Recent advances in the synthesis of nicotine and its derivatives, 20 Aug. 2007, Pages 8065-8082). It may be formulated in numerous ways suitable for the purposes of administration within this invention. Any novel means of administration suitable for the purposes of this invention are intended to be within the scope of this invention. In particular, it may be formulated in a single formulation with an alpha7 agonist described herein, or it may be formulated to be administered separately, yet in combination with the alpha7 agonist.

In certain embodiments, the enriched nicotine dosage may be formulated in the form of a lozenge, patch, gum, capsule, tablet, nasal spray, microtab, or inhalator. In a particular embodiment, the enriched nicotine dosage is in the form of a patch. A tobacco-free nicotine dosage may include 0.1% to 100% by weight nicotine, 7.5% to 99% by weight nicotine, 10% to 75% by weight nicotine, or 15% to 60% by weight nicotine. A TFN of the present invention is intended to deliver a blood concentration of nicotine greater than zero, but less than 1000 nmol/L, e.g., less than 500 nmol/L, e.g., less than 450 nmol/L, e.g., less than 400 nmol/L, e.g., less than 350 nmol/L, e.g., less than 300 nmol/L, e.g., less than 250 nmol/L, e.g., less than 200 nmol/L, e.g., less than 150 nmol/L, e.g., less than 100 nmol/L, e.g., less than 50 nmol/L, e.g., less than 25 nmol/L. In certain embodiments, a TFN of the present invention is intended to deliver a blood concentration of nicotine within the range of 25 to 444 nmol/L, e.g., 200 to 210 nmol/L.

Examples of known formulations of nicotine that would be useful as the enriched nicotine dosage in the form described (or as modified to achieve the methods of the invention) include, without limitation, U.S. Pat. No. 3,845,217 disclosing chewable compositions; U.S. Pat. No. 4,579,858 disclosing high-viscous nicotine nose-drop compositions; U.S. Pat. No. 5,525,351 disclosing nicotine-containing saliva-soluble gels; U.S. Pat. No. 5,656,255 disclosing low-viscous nicotine-containing compositions suitable for nasal spray administration; U.S. Pat. Nos. 4,920,989 and 4,953,572 disclosing the use of inhalation aerosol; BP 1,528,391 and BP 2,030,862 disclosing liquid aerosol formulations adapted as mouth-sprays, and devices for transdermal delivery of nicotine; UK Patent application GB 2 230 439 A describes nicotine lozenges with a shell or coating containing an oral-acting local analgesic, preferably eugenol; nicotine-compositions formulated in lozenges containing local analgesic have been disclosed in AU 662877 in which the latter agent is said to temporarily interfere with taste receptors which is said to reduce the desire to eat; WO 88/03803 discloses a chewable capsule filled with a liquid containing 0.1-10.0 mg of nicotine, together with additives for improving flavor and dispersion, including a variety of pH values to allow the subject a choice of nicotine absorption rates; BE 899037 discloses a tablet containing 0.1 to 5 mg nicotine as a base or water-soluble acid salt; Shaw (for example in GB 2 142 822 and U.S. Pat. No. 4,806,356) describes a nicotine lozenge prepared from a mixture of inert filler material, a binder, and either pure nicotine or a nicotine-containing substance by cold compression; U.S. Pat. No. 5,512,306 discloses a nicotine product for oral delivery in the form of an inclusion complex of nicotine and a cyclodextrin compound; and WO 97/42941 discloses a slowly erodible nicotine lozenge that allows delivery to the buccal mucosa over an extended period of time.

### IV. METHODS OF THE INVENTION

The present invention provides compounds and compositions for improving cognitive function through the combination and/or co-administration of a compound of the invention, e.g., a compound of formula (I), e.g., (R)-7-chloro-N-(quinuclidin-3-yl)benzo[b]thiophene-2-carboxamide, with a tobacco-free nicotine dosage (TFN). The combination can be used to improve aspects of cognitive function in subjects suffering from disorders associated with cognitive impairment such as Alzheimer's disease or schizophrenia.

In one embodiment, the invention provides a method for improving cognition and/or treating a cognitive disorder comprising co-administering to a subject a therapeutically effective amount of (1) a tobacco-free nicotine dosage; and (2) a compound of formula (I), or a pharmaceutically acceptable salt thereof,

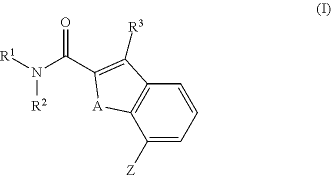

wherein

R¹ is 1-azabicyclo[2.2.2]oct-3-yl,

R² is hydrogen or (C₁-C₆)alkyl,

R³ is hydrogen, halogen or (C₁-C₆)alkyl,

A is oxygen or sulfur, and

Z is halogen, formyl, carbamoyl, cyano, trifluoromethyl, trifluoromethoxy, nitro, amino, formamido, acetamido, (C₁-C₆)alkyl, (C₁-C₆)alkyoxy, (C₁-C₆)alkylthio, (C₁-C₆)alkylamino, heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino)methyl. In one embodiment, the compound of formula (I) is (R)-7-chloro-N-(quinuclidin-3-yl)benzo[b]thiophene-2-carboxamide.

In another embodiment, the subject is treated with a compound of formula (I) wherein R² is hydrogen, R³ is hydrogen, A is sulfur, and Z is halogen, formyl, carbamoyl, cyano, trifluoromethyl, trifluoromethoxy, nitro, amino, formamido, acetamido, (C₁-C₆)alkyl, (C₁-C₆)alkyoxy, (C₁-C₆)alkylthio, or (C₁-C₆)alkylamino.

In another embodiment, the subject is treated with a compound of formula (I) wherein Z is heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino).

In another embodiment, the subject is treated with a compound of formula (I) wherein Z is halogen, cyano, trifluoromethyl, trifluoromethoxy, methyl, ethyl, methoxy, or ethoxy.

In another embodiment, the subject is treated with a compound of formula (I) wherein the compound of formula (I) is (R)-7-chloro-N-(quinuclidin-3-yl)benzo[b]thiophene-2-carboxamide.

In certain embodiments, the subject has been diagnosed with Alzheimer's disease or pre-Alzheimer's disease. In certain embodiments, the subject has been diagnosed with mild to moderate Alzheimer's disease. In certain embodiments, the subject has been diagnosed with moderate to severe Alzheimer's disease. In certain embodiments, the subject has been diagnosed with schizophrenia or schizoaffective disorder.

In any of the methods described herein, the method may improve one or more facets of cognition selected from, but not limited to learning, delayed memory, attention, working memory, visual learning, speed of processing, vigilance, verbal learning, visual motor function, social cognition, long term memory or executive function. The methods can also be used to treat: Alzheimer's disease, schizophrenia (e.g., paranoid type, disorganized type, catatonic type, and undifferentiated type), schizophreniform disorder, schizoaffective disorder, delusional disorder, positive symptoms of schizophrenia or negative symptoms of schizophrenia. In certain embodiments, the subject has been diagnosed with Alzheimer's disease or pre-Alzheimer's disease, the subject has been diagnosed with mild to moderate Alzheimer's disease, the subject has been diagnosed with moderate to severe Alzheimer's disease, the subject has been diagnosed with schizophrenia, the subject has been diagnosed with schizoaffective disorder.

In one embodiment, the methods of the invention may be used to treat disorders comprising, as a symptom thereof, a deficiency in cognition: for example, a subnormal functioning in one or more cognitive aspects such as memory, intellect, or learning and logic ability, in a particular individual relative to other individuals within the same general age population. Also, such symptoms include any reduction in any particular individual's functioning in one or more cognitive aspects, for example as occur in age-related cognitive decline. Examples of disorders that comprise as a symptom a deficiency in cognition that can be treated according to the present invention are dementia, for example Alzheimer's disease, multi-infarct dementia, alcoholic dementia or other drug-related dementia, dementia associated with intracranial tumors or cerebral trauma, dementia associated with Huntington's disease or Parkinson's disease, or AIDS-related dementia; Alzheimer's related dementia; delirium; amnestic disorder; post-traumatic stress disorder; mental retardation; a learning disorder, for example reading disorder, mathematics disorder, post operative cognitive decline, or a disorder of written expression; attention-deficit/hyperactivity disorder; and age-related cognitive decline.

In one embodiment, the methods of the invention may be used to treat anxiety or psychotic disorders such as schizophrenia, for example of the paranoid, disorganized, catatonic, undifferentiated, or residual type; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizoid type. Examples of anxiety disorders include, but are not limited to, panic disorder; agoraphobia; a specific phobia; social phobia; obsessive-compulsive disorder; post-traumatic stress disorder; acute stress disorder; and generalized anxiety disorder.

In another embodiment, the methods described herein may be used to treat Alzheimer's disease (AD), also called Alzheimer disease, senile dementia of the Alzheimer type, primary degenerative dementia of the Alzheimer's type, or simply Alzheimer's, which is the most common form of dementia. It is a degenerative disease most often diagnosed in subjects over 65 years old. Without wishing to be bound by theory, the first symptoms of Alzheimer's are often mistaken as related to aging and stress. The early symptoms can affect most daily living activities, including feeding oneself, bathing, dressing, grooming, work, homemaking, leisure activity, and memory loss. This pre-dementia period has also been termed mild Alzheimer's (a term well known in the art), or mild cognitive impairment, and includes subtle problems with executive functions of attentiveness, planning, flexibility, and abstract thinking, as well as impairments in semantic memory. Moderate AD (a term also well known in the art) is characterized by speech difficulties, impairment of reading and writing skills, and complex motor sequences becoming less coordinated making the risk of falling increasingly higher. During moderate AD, memory problems worsen, and the subject may fail to recognize close relatives. Long term memory also becomes impaired. Moderate AD often leads to advanced, or severe AD (both terms well known in the art), where the subject is completely dependent on caregivers. Language is reduced to simple phrases or even single words, eventually leading to complete loss of speech. Despite the loss of verbal language abilities, subjects can often understand and return emotional signals. Although aggressiveness can still be present, extreme apathy and exhaustion are much more common results. Subjects will ultimately not be able to perform even the most simple tasks without assistance. Muscle mass and mobility deteriorate to the point where they are bedridden, and they lose the ability to feed themselves.

In another embodiment, the methods described herein may be used to treat psychosis, which is characterized as a mental disorder characterized by gross impairment in the subject's perception of reality. The subject may suffer from delusions, and hallucinations, and may be incoherent in speech. His behavior may be agitated and is often incomprehensible to those around him. In the past, the term psychosis has been applied to many conditions that do not meet the stricter definition given above. For example, mood disorders were named as psychoses.

In yet another embodiment, the methods described herein may be used to treat pre-senile dementia (mild cognitive impairment), which is characterized by memory impairment rather than attention deficit problems, and with otherwise unimpaired cognitive functioning. Mild cognitive impairment is distinguished from senile dementia in that mild cognitive impairment involves a more persistent and troublesome problem of memory loss for the age of the subject.

In another embodiment, the methods described herein may be used to treat senile dementia, which is not a single disease state. The conditions classified under this name frequently include cognitive and attention deficits.

In certain embodiments, the methods of the present invention may be used to treat the cognitive and attention deficits as well as the neurodegeneration associated with attention deficit disorder, attention deficit hyperactivity disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, depression, general anxiety disorder, age-related macular degeneration, Parkinson's disease, tardive dyskinesia, Pick's disease, post traumatic stress disorder, dysregulation of food intake including bulimia and anorexia nervosa, withdrawal symptoms associated with smoking cessation and dependant drug cessation, Gilles de la Tourette's Syndrome, glaucoma, or symptoms associated with pain.

Both the alpha7 nicotinic acid receptor agonist (e.g., compound of formula (I)) and the enriched nicotine dosage can be administered in any convenient manner, e.g., orally, or transdermally. In some cases the compound of formula (I) is administered at a daily dose that but for the combination with the enriched nicotine dosage, would not cause a statistically significant improvement in cognition. Additionally, the combination of the compound of formula (I), and a tobacco-free nicotine dosage may provide a benefit for a wider range or subjects and/or over a longer period of treatment than either the compound of Formula (I) or the enriched nicotine dosage alone. In particular embodiments, the (R)-7-chloro-N-(quinuclidin-3-yl)benzo[b]thiophene-2-carboxamide is administered at a dose that is therapeutically effective in the absence of a tobacco-free nicotine dosage. In another particular embodiment, one or both of the (R)-7-chloro-N-(quinuclidin-3-yl)benzo[b]thiophene-2-carboxamide and a tobacco-free nicotine dosage is administered at a subclinical dose. In particular embodiments, the (R)-7-chloro-N-(quinuclidin-3-yl)benzo[b]thiophene-2-carboxamide is administered at a dose that is not completely therapeutically effective, i.e., possesses partial activity, in the absence of a tobacco-free nicotine dosage.

In this respect, it has been found that (R)-7-chloro-N-(quinuclidin-3-yl)benzo[b]thiophene-2-carboxamide can have procognitive effects in humans at unexpectedly low doses. Thus, (R)-7-chloro-N-(quinuclidin-3-yl)benzo[b]thiophene-2-carboxamide and pharmaceutically acceptable salts thereof can be used at unexpectedly low doses improve cognition in individuals suffering from impaired cognition and in healthy individuals (i.e., individuals that are not suffering from an apparent cognitive deficit). For example, it can be used to improve cognition in subjects suffering from Alzheimer's disease, schizophrenia and other disorders such as other neurodegenerative diseases (e.g., Huntington's Disease or Parkinson's Disease) and attention deficit disorder. It can be used to treat certain disorders, e.g., Alzheimer's disease, schizophrenia (e.g., paranoid type, disorganized type, catatonic type, and undifferentiated type), schizophreniform disorder, schizoaffective disorder, delusional disorder, positive symptoms of schizophrenia, negative symptoms of schizophrenia at a daily dose of 3 mg, 2.70 mg, 2.50 mg, 2.25 mg, 2 mg, 1.75 mg, 1.50 mg, 1.25 mg, 1 mg, 0.7, 0.5, 0.3 mg or even 0.1 mg. The compound can be used to improve one or more aspects of cognition, e.g., one or more of: executive function, memory (e.g., working memory), social cognition, visual learning, verbal learning and speed of processing. In a particular embodiment, (R)-7-chloro-N-(quinuclidin-3-yl)benzo[b]thiophene-2-carboxamide or a pharmaceutically acceptable salt thereof is administered at 0.03 to 1.0 mg/day. In a particular embodiment, (R)-7-chloro-N-(quinuclidin-3-yl)benzo[b]thiophene-2-carboxamide or a pharmaceutically acceptable salt thereof is orally administered at 0.03 to 0.5 mg/day. In a particular embodiment, (R)-7-chloro-N-(quinuclidin-3-yl)benzo[b]thiophene-2-carboxamide or a pharmaceutically acceptable salt thereof is orally administered at 0.03 to 0.3 mg/day. In a particular embodiment, (R)-7-chloro-N-(quinuclidin-3-yl)benzo[b]thiophene-2-carboxamide or a pharmaceutically acceptable salt thereof is orally administered at 0.03 to 0.1 mg/day.

As disclosed herein, individual components of the combinations useful in this invention may generally be administered separately, each by its own customary and known route; and in certain cases the routes of administration may be different. In a particular embodiment, administration will generally be timed so that both the alpha7 agonist, e.g., a compound of formula (I), and the enriched nicotine dosage both coincide, or nearly coincide, in reaching their maximum pharmacokinetic effect. The routes of administration can be any of those known to the art such as oral, parenteral via injection, or transdermal as by applying the active component in a gel, a patch, or other such formulation topically. Each component can be formulated as known in the art, usually together with a pharmaceutically acceptable vehicle, diluent or carrier, for example as a tablet, capsule, lozenge, troche, elixir, solution, or suspension for oral administration, in a suitable injectable vehicle for parenteral administration, or as a lotion, ointment or cream for topical application. In a particular embodiment, the compound of formula (I) and the enriched nicotine dosage are each co-administered orally, together or separately. In another particular embodiment, the enriched nicotine dosage is administered as a patch for transdermal administration.

The exact dose of each component administered will, of course, differ depending on the specific components prescribed, on the subject being treated, on the severity of the disorder, on the manner of administration and on the judgment of the prescribing physician. Thus, because of subject-to-subject variability, the dosages given below are a guideline and the physician may adjust doses of the compounds to achieve the treatment that the physician considers appropriate for the subject, male or female. In considering the degree of treatment desired, the physician must balance a variety of factors such as the age of the subject and the presence of other diseases or conditions. In general, a compound of formula (I) can be used at low doses, for example at a daily oral dose of (or no more than): 3 mg, 2.70 mg, 2.50 mg, 2.25 mg, 2 mg, 1.75 mg, 1.50 mg, 1.25 mg, 1 mg, 0.7 mg, 0.5 mg, 0.3 mg, 0.1 mg, 0.5 mg or even 0.03 mg. Thus, for example, it can be administered at 0.03-1.5 mg, 0.05-1.5 mg, 0.05-1.0 mg daily dose, including 1 mg/daily, 0.5 mg/daily or 0.3 mg/daily. In some cases the dose of (R)-7-chloro-N-(quinuclidin-3-yl)benzo[b]thiophene-2-carboxamide that is administered is a subclinical dose. In terms of concentration in the blood, the nicotine delivered through the use of a tobacco-free nicotine dosage can be administered so as to achieve a serum concentration of 1000 nmol/L, e.g., less than 500 nmol/L, e.g., less than 450 nmol/L, e.g., less than 400 nmol/L, e.g., less than 350 nmol/L, e.g., less than 300 nmol/L, e.g., less than 250 nmol/L, e.g., less than 200 nmol/L, e.g., less than 150 nmol/L, e.g., less than 100 nmol/L, e.g., less than 50 nmol/L, e.g., less than 25 nmol/L. In certain embodiments, a TFN of the present invention is intended to deliver a blood concentration of nicotine within the range of 25 to 444 nmol/L, e.g., 200 to 210 nmol/L.\

### Additional Combinations

It has also been surprisingly found that memory can be improved when (R)-7-chloro-N-(quinuclidin-3-yl)benzo[b]thiophene-2-carboxamide is administered at a subclinical dose (i.e., a dose that but for the combination would not improve memory or cognition) in combination with an acetylcholinesterase inhibitor that is also administered at a subclinical dose. Thus, a subject can experience a benefit (e.g., improved memory or cognition) from a combination of drugs each of which is administered at very low, side-effect reducing or side-effect avoiding dose. Moreover, the combination of drugs may provide a benefit for a wider range or subjects and/or over a longer period of treatment. For example, while certain acetylcholinesterase inhibitors can exhibit reduce efficacy after several months of treatment, the combination may provide a longer period of efficacy.

Accordingly, the present invention provides for the further combination of an alpha7 agonist described herein (e.g., (R)-7-chloro-N-(quinuclidin-3-yl)benzo[b]thiophene-2-carboxamide), a nicotine enriched dosage, and an acetylcholinesterase inhibitor, wherein either the alpha7 agonist, the acetylcholinesterase inhibitor, or both are administered at a subclinical dose). In various cases: the subject has been diagnosed with Alzheimer's disease or pre-Alzheimer's disease, the subject has been diagnosed with mild to moderate Alzheimer's disease, or the subject has been diagnosed with moderate to severe Alzheimer's disease. In particular embodiments, the acetylcholinesterase inhibitor is selected from tacrine, donepezil, rivastigmine and galantamine, e.g., donepezil. In certain embodiments wherein the acetylcholinesterase inhibitor is selected from donepezil, rivastigmine and galantamine, the subject has been administered an acetylcholinesterase inhibitor for a period of time prior to being administered (R)-7-chloro-N-(quinuclidin-3-yl)benzo[b]thiophene-2-carboxamide or a pharmaceutically acceptable salt thereof, wherein the prior administration has been for at least one month, e.g., the prior administration has been for at least three months, e.g., the prior administration has been for at least six months. In certain embodiments the method improves one or more of: learning, delayed memory, attention, working memory, visual learning, speed of processing, vigilance, verbal learning, visual motor function, social cognition, long term memory or executive function.

In particular embodiments, (R)-7-chloro-N-(quinuclidin-3-yl)benzo[b]thiophene-2-carboxamide or a pharmaceutically acceptable salt thereof is orally administered at 1.0 mg/day; 0.5 mg/day; 0.3 mg/day; or 0.1 mg/day. In particular embodiments, the acetylcholinesterase inhibitor is donepezil and is orally administered at 5 mg/day; 4.5 mg/day; 4.0 mg/day; 2.5 mg/day; 1.5 mg/day or less; 1.0 mg/day; and the acetylcholinesterase inhibitor is administered at a dose that achieves 10-65% steady state red blood cell acetylcholinesterase inhibition.

### V. PHARMACEUTICAL COMPOSITIONS OF THE COMBINATIONS OF THE INVENTION

As previously disclosed, the combination of an alpha7 agonist of the invention, e.g., a compound of formula (I), and enriched nicotine dosage can be administered as a composition. Thus, the compounds of this invention can be administered together in any conventional oral, parenteral, rectal or transdermal dosage form, usually also together with a pharmaceutically acceptable vehicle, diluent or carrier.

In one embodiment, the present invention provides a pharmaceutical composition comprising (R)-7-chloro-N-(quinuclidin-3-yl)benzo[b]thiophene-2-carboxamide or a pharmaceutically acceptable salt thereof and a tobacco-free nicotine dosage (TFN).

In another embodiment, the present invention provides a daily unit dosage pharmaceutical composition comprising 0.03 to 1.0 mg of (R)-7-chloro-N-(quinuclidin-3-yl)benzo[b]thiophene-2-carboxamide or a pharmaceutically acceptable salt thereof, a TFN, and a pharmaceutically acceptable carrier. In certain embodiments, the daily unit dosage pharmaceutical composition comprises 0.03 to 0.5 mg of (R)-7-chloro-N-(quinuclidin-3-yl)benzo[b]thiophene-2-carboxamide or a pharmaceutically acceptable salt thereof. In certain embodiments, the daily unit dosage pharmaceutical composition comprises comprising 0.03 to 0.3 mg of (R)-7-chloro-N-(quinuclidin-3-yl)benzo[b]thiophene-2-carboxamide or a pharmaceutically acceptable salt thereof. In certain embodiments, the daily unit dosage pharmaceutical composition comprises 0.03 to 0.1 mg of (R)-7-chloro-N-(quinuclidin-3-yl)benzo[b]thiophene-2-carboxamide or a pharmaceutically acceptable salt thereof.

Another embodiment of the present invention provides a packaged pharmaceutical comprising a package containing a unit dosage pharmaceutical composition comprising (R)-7-chloro-N-(quinuclidin-3-yl)benzo[b]thiophene-2-carboxamide or a pharmaceutically acceptable salt thereof, and a transdermal patch comprising a tobacco-free nicotine dosage. In one embodiment, the packaged pharmaceuticals may comprise a package containing a first unit dosage pharmaceutical composition comprising (R)-7-chloro-N-(quinuclidin-3-yl)benzo[b]thiophene-2-carboxamide or a pharmaceutically acceptable salt thereof, and a second unit dosage pharmaceutical composition comprising a tobacco-free nicotine dosage. In some cases the unit dosage form of nicotine is a transdermal patch, an oral form (e.g., gum) or a nasal spray. In some cases the unit dosage form of (R)-7-chloro-N-(quinuclidin-3-yl)benzo[b]thiophene-2-carboxamide or a pharmaceutically acceptable salt thereof is a tablet or capsule. The two agents can have the same or different unit dosage forms. In particular embodiments, the package further comprises instructions for use, e.g., describing a part regimen of treatment using the alpha7 agonist and enriched nicotine dosage, and such instructions may optionally form an integrated component of the packaging.

In certain embodiments, the pharmaceutical composition may also comprise an acetylcholinesterase inhibitor. As such, a particular embodiment, the present invention provides a pharmaceutical composition comprising (R)-7-chloro-N-(quinuclidin-3-yl)benzo[b]thiophene-2-carboxamide or a pharmaceutically acceptable salt thereof, a tobacco-free nicotine dosage, and an acetylcholinesterase inhibitor. In certain embodiments, the acetylcholinesterase inhibitor is selected from tacrine, donepezil, rivastigmine and galantamine, e.g., donepezil.

Also described is a daily unit dosage pharmaceutical composition comprising no more than 1.0 mg of (R)-7-chloro-N-(quinuclidin-3-yl)benzo[b]thiophene-2-carboxamide or a pharmaceutically acceptable salt thereof, a tobacco-free nicotine dosage, an acetylcholinesterase inhibitor and a pharmaceutically acceptable carrier. In various cases the daily unit dosage pharmaceutical composition comprises no more than 0.5 (0.3, or 0.1) mg of (R)-7-chloro-N-(quinuclidin-3-yl)benzo[b]thiophene-2-carboxamide or a pharmaceutically acceptable salt thereof. In certain embodiments, the daily unit dosage pharmaceutical composition comprises no more than 5, 4, 3, 2, 1, or 0.5 mg of donepezil.

For oral administration, a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; in particular embodiments, such materials also include lactose or milk sugar as well as high molecular weight polyethylene glycols. When aqueous suspensions and/or elixirs are desired for oral administration, the compounds/components of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, may be prepared.

Methods of preparing various pharmaceutical compositions with a certain amount of active ingredient are known, or will be apparent in light of this disclosure, to those of ordinary skill in this art. For examples of methods of preparing pharmaceutical compositions, see Remington's Pharmaceutical Sciences, Mack Publishing Company, Easter, Pa., 15th Edition (1975).

### EXEMPLIFICATION

The present invention is illustrated by the following examples, which are not intended to be limiting in any way.

Compounds of the invention may be synthesized according to art-recognized techniques and synthetic strategies.

### Example 1

### General Safety and Efficacy Assessment

The safety and efficacy for the combinations of the invention may be assessed in accordance with existing strategies for such assessment. For example, the safety and efficacy of the combination of (R)-7-chloro-N-(quinuclidin-3-yl)benzo[b]thiophene-2-carboxamide HCl salt in combination with a tobacco-free nicotine dosage can be assessed in subjects, e.g., subjects suffering from Alzheimer's disease or schizophrenia as follows. Subjects are dosed with placebo or one or both of (R)-7-chloro-N-(quinuclidin-3-yl)benzo[b]thiophene-2-carboxamide HCl salt and nicotine at appropriate doses. Safety can be evaluated by adverse events, ECG, and clinical laboratory measures. Cognitive effects can be measured by CogState computerized cognitive testing and all or a subset of NTB scales (e.g., category fluency, Trails A and B).

### Example 2

### Effect on Cognition in Schizophrenia Subjects

The studies described below will demonstrate that (R)-7-chloro-N-(quinuclidin-3-yl)benzo[b]thiophene-2-carboxamide hydrochloride in combination with a tobacco-free nicotine dosage form can improve sensory electrophysiological responses which correlate with improved cognitive and functional performance in schizophrenia subjects in manner that is greater than (R)-7-chloro-N-(quinuclidin-3-yl)benzo[b]thiophene-2-carboxamide hydrochloride alone. These effects are expected at a daily dose as low as 0.3 mg of (R)-7-chloro-N-(quinuclidin-3-yl)benzo[b]thiophene-2-carboxamide hydrochloride.

Impairment of the ability of central nervous system to inhibit irrelevant sensory information has long been used as a model for understanding the deficits of attention seen in schizophrenic subjects. Two approaches to the measurement of this ability have commonly been employed (see (Heinrichs, 2004; Potter et al., 2006; Turetsky et al., 2007; Umbricht and Krljes, 2005) for reviews and meta-analyses): (1) the sensory gating paradigm in which the presentation of one stimulus normally suppresses the response elicited by a stimulus which rapidly follows it. Schizophrenic subjects typically exhibit less suppression (gating) of the second response. (2) the oddball or orienting paradigm in which a rare or unexpected event elicits a diminished response in schizophrenic subjects because attentional resources are inappropriately focused on less salient aspects of the environment.

Two responses are commonly used assess brain activity: (1) the auditory P50 response elicited by the second member of a pair of clicks; and (2) the mismatch negativity (MMN) or N2 response evoked by a rarely occurring pure tone of no instructed relevance to the subject. Abnormalities in both P50 gating and the MMN have been reported in schizophrenic subjects.

The neurobiology of P50 sensory gating is well documented in studies of human and animal subjects. Its regulation relies heavily on the integrity of the hippocampus and pathways that provide input to the hippocampus (Adler et al., 1998). For example, lesions of the cholinergic pathway originating in the medial septal nucleus disrupt the gating response, as do antagonists of low affinity nicotinic receptors. Cholinergic agonists, including nicotine itself (Adler et al., 1993; Duncan et al., 2001), have been shown to enhance P50 gating (Freedman et al, 2001; Olincy et al., 2006).

The neurobiology of the MMN is more complex. Imaging studies suggest that the primary and secondary auditory cortices in the temporal lobe are important for its generation (Naatanen and Alho, 1995). The dorsolateral prefrontal cortex also contributes (Schall et al., 2003). The neurotransmitter systems underlying the MMN are understudied and largely unknown. Yet, as is the case for P50, nicotinic cholinergic systems appear important (Baldeweg et al., 2006; Dunbar et al., 2007).

The sensitivity of P300 and NIOO to cholinergic compounds has been known for many years (Dierks et al., 1994; Kaga et al., 1992). Various cholinergic antagonists—such as scopolamine—profoundly reduce the amplitudes of these components. In contrast, the components are markedly improved in amplitude by cholinesterase inhibitors (Katada et al., 2003; Werber et al., 2001) and other compounds that enhance cholinergic activity (Easton and Bauer, 1997).

The tests described above are used to study the effect of the test combination, in comparison to the alpha7 agonist alone, on cognition in subjects suffering from schizophrenia. Prior to testing in the assays described above, the subjects are dosed with a tobacco-free nicotine dosage form that would effect a blood concentration of nicotine ranging from 25 to 1000 nmol/L and 1 mg of the alpha7 agonist daily, 0.3 mg of the alpha7 agonist daily or placebo for 20 days.

### Example 3

### Effect on Cognition in Normal Subjects

The impact of an alpha7 agonist, e.g., (R)-7-chloro-N-(quinuclidin-3-yl)benzo[b]thiophene-2-carboxamide hydrochloride, in combination with a tobacco-free nicotine dosage form on cognition in normal subjects may be assessed as described below. In these studies subjects are treated with the alpha7 agonist dissolved in cranberry juice and the nicotine enriched dosage administered transdermally by means of a patch.

The impact of the combination on cognition in normal subjects is assessed in an SAD (Single Ascending Dose) study with the Digit Symbol Substitution Test (DSST). Positive effects of a test compound in the DSST indicate a beneficial effect on working memory and executive function.

In the MAD (Multiple Ascending Dose) studies cognition is assessed using tests from the CogState battery (cogstate.com). The CogState battery is a proprietary computerized cognitive battery of tests measure various cognitive domains including: attention, identification capability, working memory, visual memory, and executive function. In these studies a test combination is evaluated for an impact on: visual motor skills, learning, executive function, and delayed memory. A therapeutic profile showing pro-cognitive effects on nonverbal learning and memory and executive function without a central stimulatory effect would indicate that the drug may be very beneficial in treating subjects that have, as a feature of their condition, symptoms of anxiety or agitation.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents were considered to be within the scope of this invention and are covered by the following claims. Moreover, any numerical or alphabetical ranges provided herein are intended to include both the upper and lower value of those ranges. In addition, any listing or grouping is intended, at least in one embodiment, to represent a shorthand or convenient manner of listing independent embodiments; as such, each member of the list should be considered a separate embodiment.

1. A method for improving cognition and/or treating a cognitive disorder comprising co-administering to a subject a therapeutically effective amount of (1) a tobacco-free nicotine dosage; and (2) a compound of formula (I), or a pharmaceutically acceptable salt thereof, 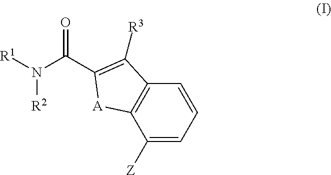
wherein
R¹ is 1-azabicyclo[2.2.2]oct-3-yl,
R² is hydrogen or (C₁-C₆)alkyl,
R³ is hydrogen, halogen or (C₁-C₆)alkyl,
A is oxygen or sulfur, and
Z is halogen, formyl, carbamoyl, cyano, trifluoromethyl, trifluoromethoxy, nitro, amino, formamido, acetamido, (C₁-C₆)alkyl, (C₁-C₆)alkyoxy, (C₁-C₆)alkylthio, (C₁-C₆)alkylamino, heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino)methyl.

2. The method according to claim 1 wherein R² is hydrogen, R³ is hydrogen, A is sulfur, and Z is halogen, formyl, carbamoyl, cyano, trifluoromethyl, trifluoromethoxy, nitro, amino, formamido, acetamido, (C₁-C₆)alkyl, (C₁-C₆)alkyoxy, (C₁-C₆)alkylthio, or (C₁-C₆)alkylamino.

3. The method according to claim 1 wherein Z is heteroaryl-carbonylamino, arylcarbonylamino, (C₁-C₄)alkylsulfonylamino, di(arylsulfonyl)amino, (C₃-C₆)cycloalkylcarbonylmethyl or amino(hydroxyimino).

4. The method according to claim 1 wherein Z is halogen, cyano, trifluoromethyl, trifluoromethoxy, methyl, ethyl, methoxy, or ethoxy.

5. The method according to claim 1 wherein the compound of formula (I) is (R)-7-chloro-N-(quinuclidin-3-yl)benzo[b]thiophene-2-carboxamide.

6. The method of claim 1 wherein the subject has been diagnosed with Alzheimer's disease or pre-Alzheimer's disease.

7. The method of claim 1 wherein the subject has been diagnosed with mild to moderate Alzheimer's disease.

8. The method of claim 1 wherein the subject has been diagnosed with moderate to severe Alzheimer's disease.

9. The method of claim 1 wherein the subject has been diagnosed with schizophrenia or schizoaffective disorder.

10. The method of claim 1, wherein the method improves one or more of: learning, delayed memory, attention, working memory, visual learning, speed of processing, vigilance, verbal learning, visual motor function, social cognition, long term memory or executive function.

11. The method of claim 1 wherein the (R)-7-chloro-N-(quinuclidin-3-yl)benzo[b]thiophene-2-carboxamide is administered at a dose that is therapeutically effective in the absence of a tobacco-free nicotine dosage.

12. The method of claim 1 wherein one or both of the (R)-7-chloro-N-(quinuclidin-3-yl)benzo[b]thiophene-2-carboxamide and a tobacco-free nicotine dosage is administered at a subclinical dose.

13. The method of claim 1 wherein (R)-7-chloro-N-(quinuclidin-3-yl)benzo[b]thiophene-2-carboxamide or a pharmaceutically acceptable salt thereof is administered at 0.03 to 1.0 mg/day.

14. The method of claim 1 wherein (R)-7-chloro-N-(quinuclidin-3-yl)benzo[b]thiophene-2-carboxamide or a pharmaceutically acceptable salt thereof is orally administered at 0.03 to 0.5 mg/day.

15. The method of claim 1 wherein (R)-7-chloro-N-(quinuclidin-3-yl)benzo[b]thiophene-2-carboxamide or a pharmaceutically acceptable salt thereof is orally administered at 0.03 to 0.3 mg/day.

16. The method of claim 1 wherein (R)-7-chloro-N-(quinuclidin-3-yl)benzo[b]thiophene-2-carboxamide or a pharmaceutically acceptable salt thereof is orally administered at 0.03 to 0.1 mg/day.

17. A pharmaceutical composition comprising (R)-7-chloro-N-(quinuclidin-3-yl)benzo[b]thiophene-2-carboxamide or a pharmaceutically acceptable salt thereof and a tobacco-free nicotine dosage (TFN).

18. A daily unit dosage pharmaceutical composition comprising 0.03 to 1.0 mg of (R)-7-chloro-N-(quinuclidin-3-yl)benzo[b]thiophene-2-carboxamide or a pharmaceutically acceptable salt thereof, a TFN, and a pharmaceutically acceptable carrier.

19. The daily unit dosage pharmaceutical composition of claim 18 comprising 0.03 to 0.5 mg of (R)-7-chloro-N-(quinuclidin-3-yl)benzo[b]thiophene-2-carboxamide or a pharmaceutically acceptable salt thereof.

20. The daily unit dosage pharmaceutical composition of claim 18 comprising 0.03 to 0.3 mg of (R)-7-chloro-N-(quinuclidin-3-yl)benzo[b]thiophene-2-carboxamide or a pharmaceutically acceptable salt thereof.

21. The pharmaceutical composition of claim 18 comprising 0.03 to 0.1 mg of (R)-7-chloro-N-(quinuclidin-3-yl)benzo[b]thiophene-2-carboxamide or a pharmaceutically acceptable salt thereof.

22. A packaged pharmaceutical comprising a package containing a unit dosage pharmaceutical composition comprising (R)-7-chloro-N-(quinuclidin-3-yl)benzo[b]thiophene-2-carboxamide or a pharmaceutically acceptable salt thereof, and a transdermal patch comprising a tobacco-free nicotine dosage.
